(19) United States
(12) Patent Application Publication
(10) Pub. No.: US 2025/0121404 A1
(43) Pub. Date: Apr. 17, 2025

(54) ELONGATED MEDICAL DEVICE, AND METHOD FOR PRODUCING ELONGATED MEDICAL DEVICE

(71) Applicant: Asahi Intecc Co., Ltd., Seto-shi (JP)

(72) Inventor: Soichi FUTAMI, Seto-shi (JP)

(73) Assignee: Asahi Intecc Co., Ltd., Seto-shi (JP)

(21) Appl. No.: 18/999,497

(22) Filed: Dec. 23, 2024

Related U.S. Application Data
(63) Continuation of application No. PCT/JP2023/021389, filed on Jun. 8, 2023.

(30) Foreign Application Priority Data
Jun. 28, 2022 (JP) 2022-103316

(51) Int. Cl. B05D 3/00

(52) U.S. Cl. CPC B05D 3/002; B05D 2503/00; B05D 2505/00

(57) ABSTRACT

The disclosed embodiments provide an elongated medical appliance including a substrate, a base layer, and a top layer, in which the top layer is composed of a polymer material with a copolymer including a polymer unit having a hydrophilic structure and crosslinked by any of the following formulas (1) to (3):

*—CH(OH)—CH(R¹)—O—C(═O)—NH—R²—NH—C(═O)—OCH(R¹)—CH(OH)—* (1)

*—CH(OH)—CH(R¹)—O—C(═O)—NH—R²—NH—C(═O)—OCH(CH(R¹)—OH)—* (2)

*—CH(CH(R¹)—OH)—O—C(═O)—NH—R²—NH—C(═O)—OCH(CH(R¹)—OH)—* (3)

and in which the base layer is composed of an isocyanate compound, and the isocyanate compound binds to the structures represented by formulas (1) to (3). This elongated medical appliance is excellent in lubricity and adhesiveness between the substrate and a coating film.

### CROSS-REFERENCE TO RELATED APPLICATIONS

The present application is a continuation of PCT/JP2023/021389, filed on Jun. 8, 2023, which claims priority to Japanese Patent Application 2022-103316, filed on Jun. 8, 2022, the entire contents of which are incorporated herein by reference.

### TECHNICAL FIELD

The disclosed embodiments relate to an elongated medical device, and a method for producing the elongated medical device.

### BACKGROUND

An elongated medical device or appliance such as a guide wire, a stent, or a catheter, which is inserted into a living body, needs to have lubricity for living body tissues, in order to prevent the living body tissues such as blood vessels with which the elongated medical appliance comes into contact in the living body from being damaged and to improve the operability of the elongated medical apparatus.

Thus, a coating film made of a hydrophilic polymer or the like has been formed on a surface of an elongated medical appliance as described above. For example, Patent Literature 1 discloses a medical equipment in which a surface lubricating layer including (a) a hydrophilic polymer having at least one reactive functional group selected from a group consisting of an epoxy group, an acid chloride group, and an aldehyde group, and (b) an antithrombotic material having a functional group capable of binding to the hydrophilic polymer (a) is provided on a surface of a substrate.

### CITATION LIST

### Patent Literature

Patent Literature 1: Japanese Patent No. 6495241

### SUMMARY

The disclosed embodiments provide an elongated medical appliance including a substrate, a base layer provided on the substrate, and a top layer on the base layer and on the side opposite to the substrate, in which the top layer includes a polymer material with a copolymer including a polymer unit having a hydrophilic structure and crosslinked by a structure represented by any of the following formulas (1) to (3).

*—CH(OH)—CH(R¹)—O—C(═O)—NH—R²—NH—C(═O)—OCH(R¹)—CH(OH)—* (1)

*—CH(OH)—CH(R¹)—O—C(═O)—NH—R²—NH—C(═O)—OCH(CH(R¹)—OH)—* (2)

*—CH(CH(R¹)—OH)—O—C(═O)—NH—R²—NH—C(═O)—OCH(CH(R¹)—OH)—* (3)

In formulas (1) to (3), * is a linking point to an adjacent atom, each R¹ is the same as or different from each other, and represents a hydrogen atom, a linear alkyl group having 1 or more carbon atoms, or a branched alkyl group having 3 or more carbon atoms. Each R² is the same as or different from each other, and represents an alkylene group having 1 or more carbon atoms, a divalent cycloaliphatic hydrocarbon group having 3 or more carbon atoms and including a cycloaliphatic structure, or a divalent aromatic group having 6 or more carbon atoms and including an aromatic ring structure. In an implementation, the alkylene group, the cycloaliphatic hydrocarbon group, or the aromatic group may optionally include a divalent group represented by —NR³— between carbon atoms, in which R³ is hydrogen atom or an alkyl group having 1 to 8 carbon atoms. The base layer includes an isocyanate compound having two or more isocyanate groups, and at least some of the isocyanate groups of the isocyanate compound react with at least some of hydroxy groups in the structures represented by the above formulas (1) to (3) in the polymer material and are covalently bonded thereto.

### Effects

The elongated medical appliance according to the disclosed embodiments includes the top layer on the surface of the substrate, and the top layer is made of a polymer material having a hydrophilic structure, so that excellent lubricity for living body tissues can be exhibited. The base layer is provided between the substrate and the top layer, and the base layer is made of an isocyanate compound that forms a covalent bond with the polymer material, so that the substrate and the top layer are sufficiently adhered to each other via the base layer to sufficiently prevent breakage, exfoliation, depletion, or the like of the top layer.

### BRIEF DESCRIPTION OF DRAWINGS

FIG. 1 is a schematic sectional view of a medical appliance according to one of the disclosed embodiments.

FIG. 2 is a diagram illustrating a state in which a crosslinked structure is formed by a reaction between a copolymer and a polyamine compound.

FIG. 3 is a diagram presenting a result of an adhesiveness test.

FIG. 4 is a diagram presenting a result of an adhesiveness test.

### EMBODIMENTS

Hereinafter, the disclosed embodiments will be explained.

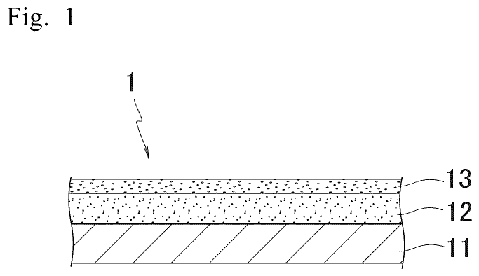

FIG. 1 is a schematic sectional view of an elongated medical appliance according to one of the disclosed embodiments.

As illustrated in FIG. 1, an elongated medical appliance 1 according to this embodiment includes a substrate 11, a base layer 12 on the substrate 11, and a top layer 13 on the base layer 12 and on the side opposite to the substrate 11.

The top layer 13 is composed of or includes a polymer material with a copolymer including a polymer unit having a hydrophilic structure and being crosslinked by a structure represented by any of the following formulas (1) to (3):

*—CH(OH)—CH(R¹)—O—C(═O)—NH—R²—NH—C(═O)—OCH(R¹)—CH(OH)—* (1)

*—CH(OH)—CH(R¹)—O—C(═O)—NH—R²—NH—C(═O)—OCH(CH(R¹)—OH)—* (2)

*—CH(CH(R¹)—OH)—O—C(═O)—NH—R²—NH—C(═O)—OCH(CH(R¹)—OH)—* (3)

In formulas (1) to (3), * is a linking point to an adjacent atom, each R¹ may be the same as or different from each other, and represents hydrogen atom, a linear alkyl group having 1 or more carbon atoms, or a branched alkyl group having 3 or more carbon atoms. Each R² may be the same as or different from each other, and represents an alkylene group having 1 or more carbon atoms, a divalent cycloaliphatic hydrocarbon group having 3 or more carbon atoms and including a cycloaliphatic structure, or a divalent aromatic group having 6 or more carbon atoms and including an aromatic ring structure. In an implementation, the alkylene group, the cycloaliphatic hydrocarbon group, or the aromatic group may optionally include a divalent group represented by —NR³— between carbon atoms, in which R³ is hydrogen atom or an alkyl group having 1 to 8 carbon atoms. As used herein, the term “or” is not necessarily an exclusive term, e.g., “A or B” would include A, B, or A and B.

The elongated medical appliance 1 according to this embodiment includes the top layer 13 made of the polymer material having the hydrophilic structure to exhibit excellent lubricity for living body tissues.

The base layer 12 is composed of or includes an isocyanate compound having two or more isocyanate groups. At least some of the isocyanate groups of the isocyanate compound react with at least some of the hydroxy groups in the structure represented by formulas (1) to (3) in the polymer material to form covalent bonds.

In the elongated medical appliance 1 according to this embodiment, the substrate 11 and the top layer 13 are laminated or attached via the base layer 12, so that the adhesiveness between the substrate 11 and the top layer 13 is sufficient, and breakage, exfoliation, depletion, or the like of the top layer 13 is favorably suppressed throughout the use of the elongated medical appliance 1 according to this embodiment.

### Components of Elongated Medical Appliance

### Top Layer

The top layer 13 according to this embodiment is composed of a polymer material with a copolymer including a polymer unit having a hydrophilic structure and crosslinked by a structure represented by one of the following formulas (1) to (3):

*—CH(OH)—CH(R¹)—O—C(═O)—NH—R²—NH—C(═O)—OCH(R¹)—CH(OH)—* (1)

*—CH(OH)—CH(R¹)—O—C(═O)—NH—R²—NH—C(═O)—OCH(CH(R¹)—OH)—* (2)

*—CH(CH(R¹)—OH)—O—C(═O)—NH—R²—NH—C(═O)—OCH(CH(R¹)—OH)—* (3)

In each formula, * is a linking point to an adjacent atom, each R¹ may be the same as or different from each other, and represents hydrogen atom, a linear alkyl group having 1 or more carbon atoms, or a branched alkyl group having 2 or more carbon atoms. The upper limit value of the number of carbon atoms in these alkyl groups may be, e.g., 4.

On the other hand, R² is an alkylene group having 1 or more carbon atoms, a divalent cycloaliphatic hydrocarbon group having 3 or more carbon atoms and including a cycloaliphatic structure, or a divalent aromatic group having 6 or more carbon atoms and including an aromatic ring structure. In an implementation, the alkylene group, the cycloaliphatic hydrocarbon group, and the aromatic group may have a divalent group represented by —NR³—, in which R³ is hydrogen atom or an alkyl group having 1 to 8 carbon atoms, between carbon atoms. The upper limit value of the number of carbon atoms in the alkylene group may be, e.g., 5. The upper limit value of the number of carbon atoms in the cycloaliphatic structure may be, e.g., 6. The number of carbon atoms in the cycloaliphatic hydrocarbon group may be, e.g., 3 to 12. The upper limit value of the number of carbon atoms in the aromatic ring structure may be, e.g., 10. The number of carbon atoms in the aromatic group may be, e.g., 6 to 20. The number of the divalent groups represented by —NR³— may be, e.g., 1 or 2. In an implementation, R² may be an alkylene group having 1 to 6 carbon atoms, e.g., an alkylene group having 4 to 6 carbon atoms.

As described above, the copolymer contains the polymer unit having the hydrophilic structure, but may contain another polymer unit. In particular, from the viewpoint of facilitating formation of the crosslinked structures of formulas (1) to (3) described above, the copolymer may further contain a polymer unit having a cyclic carbonate structure. The copolymer may be crosslinked with a crosslinking agent to form the crosslinked structures represented by the above formulas (1) to (3). In this case, from the viewpoint of facilitating formation of the crosslinked structures, the crosslinking agent may be a polyamine compound. The polymer unit having the hydrophilic structure, the polymer unit having the cyclic carbonate structure, the polyamine compound, and the like will be explained below.

### Polymer Unit Having Hydrophilic Structure

The polymer unit having the hydrophilic structure has a hydrophilic structure that makes the polymer unit hydrophilic. The hydrophilic structure may be, e.g., a neutrally charged structure. Examples of the neutrally charged hydrophilic structure include a betaine structure, an amide structure, an alkylene oxide structure, and a lactam structure. In an implementation, as the polymer unit having the hydrophilic structure, a polymer unit having a hydrophilic structure other than the betaine structure, the amide structure, the alkylene oxide structure, and the lactam structure described above may be used, and for example, a polymer unit having a charged hydrophilic structure that is not neutrally charged can be used. Each of the polymer units having the hydrophilic structure may be used alone or in combination of two or more types.

The betaine structure refers to a structure which has positive charges and negative charges at positions not adjacent to each other in the same molecule and is neutral as a whole (having no charges), in which dissociable hydrogen is not bonded to positively charged atoms. In the betaine structure, as a positively charged functional group, for example, any of quaternary ammonium, sulfonium, and phosphonium can be used, and as the negatively charged functional group, for example, any of sulfonic acid, carboxylic acid, and phosphonic acid can be used. In an implementation, the betaine structure can be, for example, a sulfobetaine, a carboxybetaine, or a phosphobetaine.

The betaine structure according to this embodiment can have positively charged functional groups and negatively charged functional groups as described above in various combinations with each other. As the betaine structure according to this embodiment, for example, a structure derived from N-methacryloylaminopropyl-N,N-dimethylammonium-α-N-methylcarboxybetain (MAMCMB), N-methacryloyloxyethyl-N,N-dimethylammonium-α-N-methylcarboxybetain (CMB), 2-methacryloyl-oxyethyl-phosphorylcholine (MPC), 3-methacryloylamino-propyl-dimethyl-3-sulfobetain (SMB), or the like can be suitably used.

When the copolymer according to this embodiment includes a polymer unit having a cyclic carbonate structure, the copolymer may be cured through crosslinking with a polyamine compound as a crosslinking agent by ring-opening of the cyclic carbonate structure in the polymer unit, as described below. In an implementation, the positively charged functional groups in the betaine structure may include a quaternary ammonium, because the quaternary ammonium can serve as a catalyst for the reaction associated with the above-described crosslinking.

The following Formula 4 represents a polymer unit derived from N-methacryloylaminopropyl-N,N-dimethylammonium-α-N-methylcarboxybetaine (MAMCMB) as the polymer unit having the betaine structure as the hydrophilic structure.

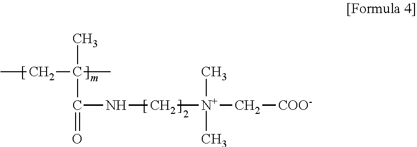

The amide structure has an amide bond. Examples of the polymer unit having an amide bond as the hydrophilic structure include polymer units derived from N,N-dimethylacrylamide (DMAAm), N-isopropylacrylamide (NiPPAM), acrylamide (AAm), methylacrylamide (MAAm), 2-acrylamide-2-methylpropylsulfonic acid (AMPS), methacrylamide, N-vinylformamide, N-vinylacetamide, N-vinylpyrrolidone, and the like. In an implementation, polymer units may be derived from N,N-dimethylacrylamide (DMAAm), N-isopropylacrylamide (NiPPAM), acrylamide (AAm), methylacrylamide (MAAm), or 2-acrylamide-2-methylpropylsulfonic acid (AMPS). The amide structures included in these polymer units may exhibit no charge ununiformity and overall neutrality.

When the copolymer according to this embodiment includes a polymer unit having a cyclic carbonate structure, the copolymer may be cured through crosslinking with a polyamine compound as a crosslinking agent by ring-opening of the cyclic carbonate structure in the polymer unit, as described below. In an implementation, the tertiary ammonium contained in the amide structure may serve as a catalyst for the reaction associated with the above-described crosslinking.

The following Formula 5 represents a polymer unit derived from N,N-dimethylacrylamide (DMAAm) as the polymer unit having the amide structure as the hydrophilic structure.

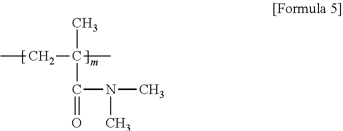

The alkylene oxide structure has an alkylene oxide group (—RO—; with the proviso that R is an alkylene group, and the number of carbon atoms in R may be 1 to 5). Examples of the polymer unit including the structure having the alkylene oxide group as the hydrophilic structure include polymer units derived from alkoxypolyalkylene glycol acrylate, alkoxypolyalkylene glycol methacrylate, alkoxyalkyl acrylate, and alkoxyalkyl methacrylate.

More specific examples of the polymer unit including the structure having the alkylene oxide group as the hydrophilic structure include polymer units derived from methoxypolyethylene glycol acrylate, methoxypolyethylene glycol methacrylate, methoxyethyl acrylate, methoxyethyl methacrylate, methoxypolypropylene glycol acrylate, methoxypolypropylene glycol methacrylate, methoxymethyl acrylate, methoxymethyl methacrylate, ethoxymethyl acrylate, ethoxymethyl methacrylate, ethoxyethyl acrylate, ethoxyethyl methacrylate, ethoxypropyl acrylate, and ethoxypropyl methacrylate. The alkylene oxide structures included in these polymer units may exhibit no charge ununiformity and overall neutrality.

The following Formula 6 represents a polymer unit derived from methoxypolyethylene glycol methacrylate (M90G) as the polymer unit having the alkylene oxide structure as the hydrophilic structure.

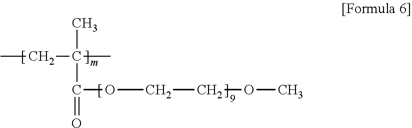

The following Formula 7 represents a polymer unit derived from methoxyethyl acrylate (MEA) as the polymer unit having the alkylene oxide structure as the hydrophilic structure.

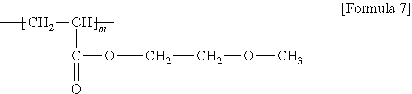

Examples of the lactam structure include a β-lactam (4-membered ring) structure, a γ-lactam (5-membered ring) structure, a δ-lactam (6-membered ring) structure, and an ε-lactam (7-membered ring) structure. In an implementation, a γ-lactam (5-membered ring) structure may be used. Examples of the polymer unit having the lactam structure as the hydrophilic structure include polymer units derived from vinyl monomers having a 5-membered ring lactam structure such as N-vinylpyrrolidone, N-vinyl-5-methylpyrrolidone, N-vinyl-5-ethylpyrrolidone, N-vinyl-5-propylpyrrolidone, N-vinyl-5-butylpyrrolidone, and 1-(2-propenyl)-2-pyrrolidone; vinyl monomers having a 6-membered ring lactam structure such as N-vinylpiperidone; and vinyl monomers having a 7-membered ring lactam structure such as N-vinylcaprolactam. The lactam structures included in these polymer units may exhibit no charge ununiformity and overall neutrality.

The following Formula 8 represents a polymer unit derived from N-vinylpyrrolidone (NVP) as the polymer unit having the lactam structure as the hydrophilic structure.

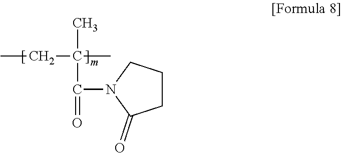

As the polymer unit having the hydrophilic structure, various hydrophilic polymer units may be used besides the above-described polymer units. Examples of such polymerized units include polymer units derived from acrylic acid and acrylates such as sodium acrylate, methacrylic acid and methacrylates such as sodium methacrylate, maleic anhydride, 2-hydroxyethyl methacrylate (HEMA), 2-hydroxyethyl acrylate (2HEA), 2-hydroxypropyl acrylate (2HPA), 2-hydroxypropylmethyl acrylate (2HPMA), 4-hydroxybutyl acrylate (4HBA), 4-hydroxybutyl methacrylate (4HBMA), 1,4-cyclohexanedimethanol monoacrylate (CHDMA), lactic acid and other amino acids, acryloylmorpholine (AMP), and N,N-dimethylaminoethyl acrylate.

The content of the polymer unit having the hydrophilic structure in the copolymer according to this embodiment may be 50 mol % or more, from the viewpoint of facilitating securance of hydrophilicity, and from the view point of facilitating acquisition of lubricity, it may be 70 mol % or more, 80 mol % or more, or 85 mol % or more. The content may be 98 mol % or less, 97 mol % or less, or 95 mol % or less. The content can also be adopted as a content of a polymer unit having a betaine structure or a polymer unit having an amide structure, as described later.

As the polymer unit having the hydrophilic structure, a polymer unit having a betaine structure may be used. In this case, the content of the polymer unit having the betaine structure in the copolymer may be 10 mol % or more, 20 mol % or more, 30 mol % or more, or 40 mol % or more from the viewpoint of facilitating acquisition of superior lubricity. Even if the polymer unit having the betaine structure is used as the polymer unit having the hydrophilic structure, it may be used in combination with a polymer unit having at least one selected from an amide structure, an alkylene oxide structure, and a lactam structure.

As the polymer unit having the hydrophilic structure, a polymer unit having an amide structure may be used. In this case, the content of the polymer unit having the amide structure in the copolymer may be 10 mol % or more, 30 mol % or more, 50 mol % or more, 70 mol % or more, 80 mol % or more, or 85 mol % or more, from the viewpoint of facilitating acquisition of superior lubricity and facilitating improvement of crosslinkability at low temperature. Even if the polymer unit having the amide structure is used as the polymer unit having the hydrophilic structure, it may be used in combination with a polymer unit having at least one selected from a betaine structure, an alkylene oxide structure, and a lactam structure.

### Polymer Unit Having Cyclic Carbonate Structure

The polymer unit having the cyclic carbonate structure has at least one cyclic carbonate group. For example, the number of the cyclic carbonate groups in the polymer unit having the cyclic carbonate structure according to this embodiment may be 1 to 3, 1 or 2, or 1.

The following Formula 9 represents an example of the “cyclic carbonate group”.

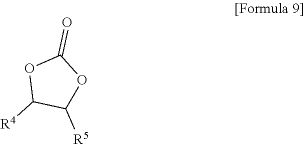

In the above Formula 9, R⁴ represents any of hydrogen atom, a linear alkyl group having 1 to 4 carbon atoms, a branched alkyl group having 1 to 4 (e.g., 3 or 4) carbon atoms, a linear alkenyl group having 1 to 4 carbon atoms, and a branched alkenyl group having 1 to 4 (e.g., 3 or 4) carbon atoms. In this formula, at least one hydrogen atom of R⁴ may be substituted or replaced with a halogen atom, and at least one carbon atom (—C—) may be substituted or replaced with —O—, —S—, or —P—. Examples of the linear or branched alkyl group having 1 to 4 carbon atoms in R⁴ include a methyl group, an ethyl group, an n-propyl group, an isopropyl group, an n-butyl group, and an isobutyl group. Examples of the linear or branched alkenyl group having 1 to 4 carbon atoms include the above-described alkyl groups in which at least one, e.g., one, of the direct carbon-carbon bonds in the alkenyl group is replaced with an unsaturated double bond. From the viewpoint of facilitating improvement of water resistance, R⁴ may be a hydrogen atom or a methyl group, e.g., a hydrogen atom.

In the above Formula 9, R⁵ represents a linear or branched alkylene group or alkenylene group having 1 to 4 carbon atoms. In this formula, at least one hydrogen atom of R⁵ may be substituted or replaced with halogen atom, and at least one carbon atom (—C—) may be substituted or replaced with —O—, —S—, or —P—. Examples of the linear or branched alkylene group having 1 to 4 carbon atoms in R⁵ include a methylene group, an ethylene group, an n-propylene group, an isopropylene group, an n-butylene group, a methylmethylene group, a methylethylene group, a dimethylethylene group, and a methylpropylene group. Examples of the linear or branched alkenylene group having 1 to 4 carbon atoms include the above-described alkylene groups in which at least one, e.g., one, of the direct carbon-carbon bonds is replaced with an unsaturated double bond. From the viewpoint of facilitating improvement of water resistance, R⁵ may be a linear or branched alkylene group having 1 to 4 carbon atoms, e.g., a linear alkylene group having 1 carbon atom (e.g., a methylene group).

In an implementation, the “cyclic carbonate group” may be a 2-oxo-1,3-dioxolane structure, e.g., a (2-oxo-1,3-dioxolane-4-yl) group.

The polymer unit having the cyclic carbonate structure may be derived from a (meth)acrylate having a cyclic carbonate group. The polymer unit may be a polymer unit derived from (meth)acrylate in which a group represented by the following Formula 10 and R⁵ of the cyclic carbonate group represented by the above Formula 9 are directly bonded to each other.

CH₂═CR⁶—R⁷—(CH₂)ₙ— [Formula 10]

In Formula 10, R⁶ represents hydrogen atom or methyl group, R⁷ represents —COO— or —CO—NH—, and n represents an integer of 1 to 4.

Specific examples of the (meth)acrylate having the cyclic carbonate group described above include (2-oxo-1,3-dioxolane-4-yl) methyl methacrylate (GCMA) and (2-oxo-1,3-dioxolane-4-yl) methyl acrylate (GCA), e.g., (2-oxo-1,3-dioxolane-4-yl) methyl methacrylate (GCMA).

The content of the polymer unit having the cyclic carbonate structure in the copolymer according to this embodiment may be 2 mol % or more, 3 mol % or more, or 5 mol % or more from the viewpoint of securing adhesiveness between the top layer 13 and the base layer 12. The content may be 50 mol % or less, 30 mol % or less, 20 mol % or less, or 15 mol % or less.

### Other Configurations of Copolymer

The copolymer according to this embodiment may include a structural unit other than the polymer unit having the hydrophilic structure and the polymer unit having the cyclic carbonate structure described above.

Examples of the other structural units include polymer units having a long-chain aliphatic structure, such as polymer units derived from n-butyl methacrylate and polymer units derived from n-lauryl methacrylate. When the copolymer includes these polymer units, a glass transition point or glass transition temperature Tg of the copolymer is appropriately lowered, and the copolymer is easily softened.

As other examples of the other structural units, it is allowed to use a polymer unit having a functional group that is crosslinkable by light irradiation, such as a polymer unit derived from 4-methacryloyloxybenzophenone (MBP) and 4-methacryloyloxy-2-hydroxybenzophenone (MHP).

The copolymer according to this embodiment may be a random copolymer or a block copolymer of the above-described polymer unit, or a mixture thereof.

The copolymer according to this embodiment has a weight-average molecular weight of 10,000 or more, or 40,000 or more. The weight-average molecular weight may be 1,000,000 or less, or 90,000 or less.

Examples of the method for producing the copolymer according to this embodiment may include, e.g., a solution polymerization method, a bulk polymerization method, an emulsion polymerization method, a suspension polymerization method. In an implementation, a solution radical polymerization method may be used.

### Polyamine Compound

The above-described polyamine compound may have two or more primary amines in its molecule. Examples of the polyamine compound include amine crosslinking agents such as aliphatic polyamine, cycloaliphatic polyamine, and aromatic polyamine.

More specific examples of the aliphatic polyamine include hexamethylenediamine (HMDA), 1,4-butanediamine (BDA), diethylenetriamine (DETA), and triethylenetetramine (TETA).

More specific examples of the aliphatic polyamine include menthenediamine (MDA) and isophoronediamine (IPDA).

More specific examples of the aromatic polyamine include metaxylenediamine (m-XDA), diaminodiphenylmethane (DDM), an m-phenylenediamine (m-PDA). In particular, hexamethylenediamine (HMDA) is a long-chain aliphatic compound that has high structural reactivity and flexibility and is suitable as a crosslinking agent. Hexamethylenediamine has lower toxicity than other diamine compounds having a shorter chain length and is suitable for medical appliance applications.

The amount of the polyamine compound serving as a crosslinking agent may be a suitable amount, as long as the polyamine compound can be crosslinked with the cyclic carbonate structure to form a desired top layer 13. The amount of the polyamine compound may be 1 to 10 equivalents, 2 to 8 equivalents, or 3 to 5 equivalents, based on 1 equivalent of the cyclic carbonate structure.

### Crosslinked Structure

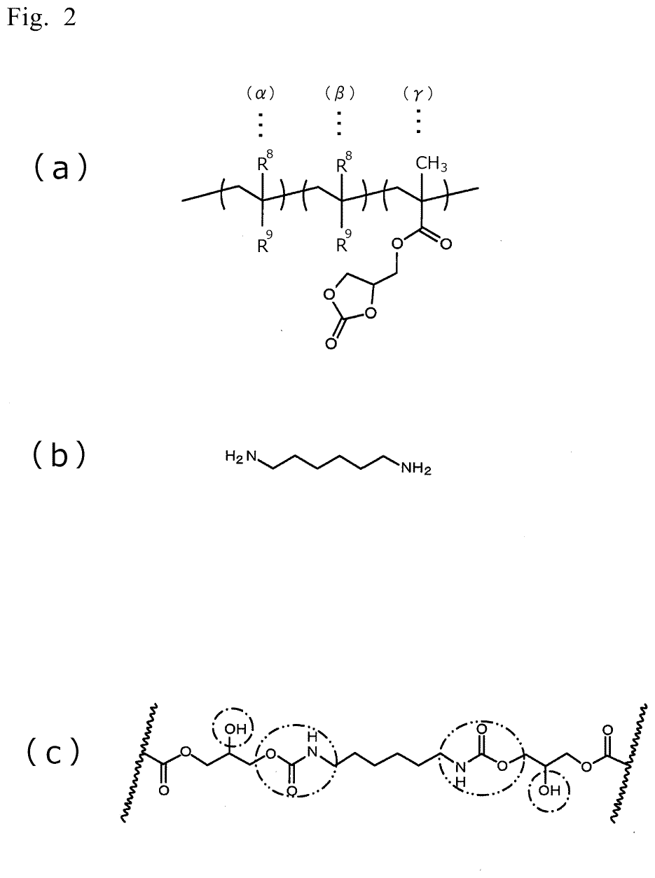

With reference to FIG. 2, a state in which the crosslinked structures of the above formulas (1) to (3) are formed by a reaction between the copolymer and the polyamine compound described above will be explained below.

Part (a) of FIG. 2 is a schematic diagram illustrating a copolymer including a polymer unit having a hydrophilic structure and a polymer unit having a cyclic carbonate structure. In the figure, although the polymer unit (α), the polymer unit (β), and the polymer unit (γ) are arranged in order, this figure does not indicate the actual arrangement of the polymer units in the copolymer but means that the copolymer is constituted such that these three polymer units are arranged at random.

In part (a) of FIG. 2, the polymer unit (α) and the polymer unit (β) each represent a polymer unit having a hydrophilic structure, and the polymer unit (γ) represents a polymer unit having a cyclic carbonate structure. In the polymer unit (α) and the polymer unit (β), R⁸ represents hydrogen atom (H) or a methyl group (CH₃), and R⁹ represents a group having a hydrophilic structure. As the polymer unit (γ), a polymer unit derived from (2-oxo-1,3-dioxolane-4-yl) methylmethacrylate (GCMA) is depicted.

Part (b) of FIG. 2 illustrates hexamethylenediamine as a specific example of the polyamine compound.

Part (c) of FIG. 2 illustrates a structure formed by crosslinking the copolymer in part (a) of FIG. 2 with the polyamine compound in part (b) of FIG. 2 (polyhydroxy urethane). In part (c) of FIG. 2, a portion (main chain of the copolymer) other than the cyclic carbonate structure in the copolymer in part (a) of FIG. 2 is simplified and represented by the wavy line. In the above-described crosslinking reaction, oxygen atom in the cyclic carbonate structure of the polymer unit (γ) is attacked by the amino group of the polyamine compound, resulting in ring-opening of the cyclic carbonate structure. At the same time, in the cyclic carbonate structure, carbon atom constituting the carbonyl group and nitrogen atom constituting the amino group covalently bind to each other to form a urethane bond (area surrounded by the two-dot chain line in part (c) of FIG. 2). Since the urethane bond is formed at each of the two amino groups of the polyamine compound, a structure with the copolymers crosslinked with each other via the polyamine compound will be formed.

Also a hydroxy group (area surrounded by a dashed-dotted line in the figure) will be generated in association with the ring-opening of the cyclic carbonate structure described above. The hydroxy group reacts with the isocyanate group of an isocyanate compound constituting the base layer 12. Thus, the polymer material constituting the top layer 13 and the isocyanate compound constituting the base layer 12 are connected to each other via a covalent bond, and the top layer 13 is firmly fixed to the base layer 12. Even if the hydroxy group is included in the polymer unit (α) or the polymer unit (β), the hydroxy group (area surrounded by the dashed-dotted line in the figure) in the crosslinked structure is preferentially used for the covalent bond with the isocyanate compound, and therefore consumption of the hydroxy groups in the polymer unit (α) and the polymer unit (β) is suppressed, resulting in high lubricity.

When the cyclic carbonate structure is ring-opened, the obtained crosslinked structure may vary depending upon which one of oxygen atoms is attacked by the amino group of the polyamine compound. That means, since the cyclic carbonate structure of the copolymer illustrated in FIG. 2 (a) has two oxygen atoms, there are two different types of post-reaction structures due to the difference in attacked oxygen atom. As a result of such a difference at both ends of the polyamine compound, three types of crosslinked structures represented by the above-described formulas (1) to (3) can be generated.

### Top Layer Composition

The method for forming the top layer 13 according to this embodiment may include forming the top layer 13 by previously preparing a top layer composition containing at least the above-described copolymer and the polyamine compound, and applying the top layer composition onto a predetermined position (surface of the base layer 11, or a base layer coat film formed by applying a base layer composition described later on the base layer), from the viewpoint of facilitating formation of the desired top layer 13.

The top layer composition may further contain a solvent so that the concentration, the viscosity, and the like of the top layer composition fall within appropriate ranges. In an implementation, a solvent may be used so that the top layer composition can be easily applied. Examples of the solvent may include alcohols such as ethanol, methanol, propanol, 2-propanol, butanol, and benzyl alcohol, as well as various hydrophilic polar solvents such as N-methyl-2-pyrrolidone (NMP), dimethyl sulfoxide (DMSO), N,N-dimethylformamide (DMF), and dimethylacetamide (DMA). In an implementation, the top layer composition may contain a polymerization initiator or a catalyst.

The amount of the solvent should be adjusted as appropriate to such an amount that facilitates the application. For example, the amount of the solvent can be 10 to 99% by mass based on the whole coating liquid of the top layer composition to be obtained.

The coat film formed by application of the top layer composition is heated to e.g. 70 to 150° C. to advance the crosslinking reaction, so that the top layer 13 can be formed. Besides the heating for the crosslinking reaction, heating for volatilizing the solvent may be previously conducted. The heating for the crosslinking reaction may double as the heating for causing the reaction between the hydroxy group of the polymer material constituting the top layer 13 and the isocyanate compound constituting the base layer 12.

### Thickness of Top Layer

The top layer 13 may have a thickness of 0.5 μm or larger, 1.0 μm or larger, or 2.0 μm or larger from the viewpoint of improving the lubricity. The top layer 13 may have a thickness of 1,000 μm or smaller, 100 μm or smaller, or 50 μm or smaller, from the viewpoint of suppressing excessive enlargement of the coating film and preventing the shape of the elongated medical appliance 1 from excessively deviating from a designed range.

### Base Layer

The base layer 12 according to this embodiment is composed of or includes an isocyanate compound having two or more isocyanate groups, as described above. At least some of the isocyanate groups of the isocyanate compound react with at least some of the hydroxy groups (hydroxy group surrounded by the dashed-dotted line in part (c) of FIG. 2) in the structure represented by formulas (1) to (3) in the above-described polymer material to form covalent bonds.

If a hydroxy group is present on the surface of the substrate 11, the hydroxy group and the isocyanate group of the isocyanate compound may also react with each other to form a covalent bond. Thereby, the adhesiveness between the substrate 11 and the base layer 12 is improved, and the base layer 12 and the top layer 13 are further prevented from, e.g., breaking and exfoliating or delaminating from the substrate 11. Even if the hydroxy group is absent on the surface of the substrate 11, the base layer 12 can sufficiently adhere to the substrate 11 through mechanical bond. That means, the base layer 12 is caught by microscopic or macroscopic irregularities on the surface of the substrate 11, resulting in sufficient adhesiveness.

The base layer 12 may contain components other than the isocyanate compound, for example, a predetermined resin. In an implementation, the base layer 12 contains the isocyanate compound as well as a resin having at least one of a hydroxy group and a carboxy group. In the resin, the hydroxy group and the carboxy group can react with the isocyanate group of the isocyanate compound to covalently bind thereto. As a result, a stronger base layer 12 can be easily formed, and breakage, exfoliation, or the like of the base layer 12 and the top layer 13 associated with use of the elongated medical appliance 1 can be more easily prevented.

### (2-1) Isocyanate Compound

The isocyanate compound may be a suitable compound that has two or more isocyanate groups. Specific examples of the isocyanate compound include aliphatic polyvalent isocyanates such as hexamethylene diisocyanate; aromatic polyvalent isocyanates such as tolylene diisocyanate, diphenylmethane diisocyanate, and xylylene diisocyanate; cycloaliphatic polyvalent isocyanates such as isophorone diisocyanate and hydrogenated diphenylmethane diisocyanate; and their biurets, isocyanurates, as well as adducts that are reaction products with a low molecular active hydrogen-containing compound such as ethylene glycol, propylene glycol, neopentyl glycol, trimethylolpropane, and castor oil.

In an implementation, a biuret of hexamethylene diisocyanate may be from the viewpoint that the biuret desirably reacts with the hydroxy group of the polymer material constituting the top layer 13 and thereby a high adhesiveness can be easily achieved.

### (2-2) Resin Having at Least One of Hydroxy Group and Carboxy Group

Examples of the resin having at least one of the hydroxy group and the carboxy group include, e.g., a (meth)acrylic resin, a polyvinyl alcohol resin, a polyhydroxyethyl methacrylate resin, a polyethylene glycol resin, an acrylic acid resin, and a maleic acid resin. The (meth)acrylic resin may be a suitable resin having a polymer unit based on a monomer having an acryloyl group (H₂C═CH—C(═O)—) or a methacryloyl group (H₂C═C(CH₃)—C(═O)—).

When the resin having at least one of the hydroxy group and the carboxy group is used in combination with the isocyanate compound, the content of the resin may be 0.1 parts by mass or more, 0.4 parts by mass or more, or 1.0 parts by mass or more based on 100 parts by mass of the isocyanate compound. The content of the resin may be 10,000 parts by mass or less, 5,000 parts by mass or less, 1,000 parts by mass or less based on 100 parts by mass of the isocyanate compound. When the content is within these ranges, the reaction between the isocyanate compound and the resin sufficiently proceeds, and a firmer base layer 12 is easily formed.

### (2-3) Others

The base layer 12 may further contain components other than the isocyanate compound and the resin having at least one of the hydroxy group and the carboxy group. As the other compounds described above, a vinyl chloride resin, a urethane elastomer, a nylon elastomer, a polyether block amide elastomer, an ethylene acrylic acid resin, an epoxy resin, or the like may be used from the viewpoint of forming a firmer base layer 12 and improving the adhesiveness between the substrate 11 and the base layer 12 and the adhesiveness between the base layer 12 and the top layer 13.

The base layer 12 may contain various catalysts for enhancing the reaction of the isocyanate group. The catalyst may be a suitable catalyst that enhances the reaction between the isocyanate group and the hydroxy group, and examples thereof include organic tin catalysts, organic bismuth catalysts, various metal complex catalysts, and amine catalysts. The catalyst may be added to any one or both of the base layer 12 and the top layer 13, or alternatively a catalytic effect may be imparted to the resin or polymer itself for forming the base layer 12 and the top layer 13.

### (2-4) Base Layer Composition

The method for forming the base layer 12 according to this embodiment may include forming the base layer 12 by previously preparing a base layer composition containing the isocyanate compound, the resin having at least one of the hydroxy group and carboxy group, and the like and subsequently applying the base layer composition onto the surface of the substrate 11, from the viewpoint of facilitating formation of the desired base layer 12.

The base layer composition may further contain a solvent so that the concentration, the viscosity, and the like of the base layer composition fall within appropriate ranges. In an implementation, a solvent for facilitating use of the base layer composition may be used. Specific examples of the solvent may include the same solvents as described above for the top layer composition. The amount of the solvent in the base layer composition is also the same as described for the top layer composition. In an implementation, the base layer composition may contain a polymerization initiator or a catalyst.

The coat film formed by application of the base layer composition is heated to e.g. 50 to 150° C. to advance the reaction between the isocyanate group and the hydroxy group, so that the base layer 12 can be formed. In addition to the heating for the reaction, heating for volatilizing the solvent may be previously conducted. The heating for the reaction may double as the heating for causing the crosslinking reaction described above for the component constituting the top layer 13.

### (2-5) Thickness of Base Layer

The base layer 12 may have a thickness of 0.1 μm or larger, 0.5 μm or larger, or 1.0 μm or larger from the viewpoint of sufficiently improving the adhesiveness between the top layer 13 and the substrate 11. The base layer 12 may have a thickness of 1,000 μm or smaller, 100 μm or smaller, or 50 μm or smaller from the viewpoint of suppressing excessive enlargement of the coating film and preventing the shape of the elongated medical appliance 1 from excessively deviating from a designed range.

### Substrate

The structure and shape of the substrate 11 according to this embodiment are selected as appropriate depending upon the type of the elongated medical appliance 1. The material constituting the substrate 11 is also selected as appropriate depending upon the type of the elongated medical appliance 1, and examples thereof include a metal material, a polymer material, and a ceramic material. Plural types of materials may be combined.

The metal material may include a suitable material that is used for an elongated medical appliance particularly to be inserted or disposed in a living body. Examples of the metal material include stainless steels such as SUS302, SUS304, and SUS316, nickel-titanium alloys, carbon steels, nickel-chromium alloys, cobalt alloys, and tungsten. These metal materials may be used alone or in combination of two or more types.

When the material constituting the substrate 11 is a metal material, the surface of the substrate 11 where the base layer 12 is provided may be subjected to a treatment for improving the adhesiveness of the base layer 12. Examples of such treatment include a phosphoric acid treatment, an ethylene-acrylic acid coating treatment, an epoxy adhesive coating treatment, an ozone/ultraviolet treatment, a plasma treatment, a corona discharge treatment, a flame treatment, and a radiation treatment.

The polymer material may be a suitable material that is used for an elongated medical appliance particularly to be inserted or disposed in a living body. Examples of the polymer material include polyamide, polyimide, modified polyolefin, polyvinyl alcohol, polyurethane, polyurea, polyester, polyether, and polylactic acid. These polymer materials may be used alone or in combination of two or more types.

Also, when the material constituting the substrate 11 is a polymer material, the surface of the substrate 11 where the base layer 12 is provided may be subjected to a treatment for improving the adhesiveness of the base layer 12. Examples of such a treatment include a primer treatment, an oxidation method, and a roughening method. Examples of the oxidation method include a corona discharge treatment, a chromic acid treatment, a flame treatment, a hot air treatment, an ozone/ultraviolet treatment, and a radiation treatment, and examples of the roughening method include a sand blasting method and a solvent treatment method.

### Elongated Medical Appliance

The type of the elongated medical appliance 1 according to this embodiment may include a suitable appliance that requires lubricity for a living body tissue. In general, the elongated medical appliance 1 may be inserted or disposed in a living body, and may be a guide wire or a catheter.

Examples of the catheter include, e.g., a guiding catheter, a penetration catheter, a microcatheter, a balloon catheter, a foreign object removal catheter, an imaging catheter, a biliary catheter, a urethral catheter, an endoscope, and a dilator.

Examples of the guide wire include, e.g., a percutaneous coronary intervention (PCI) guide wire for treating coronary arteries, a percutaneous transluminal angioplasty (PTA) guide wire for treating lower limb blood vessels, an interventional radiology (IVR) guide wire for treating peripheral blood vessels, an INR guide wire for treating cerebral blood vessels, and a coronary angiography (CAG) guide wire for imaging.

As more specific constitutional examples of the elongated medical appliance 1 according to this embodiment, various configurations as described in the following (a) to (e) can be adopted.
a) A guide wire including a linear core wire, a coating layer provided on at least a part of the outer periphery of the core wire, and the base layer 12 and the top layer 13 formed on the surface of the coating layer.
b) A guide wire including a linear core wire, a coil layer with a wire spirally wound around at least a part of the outer periphery of the core wire, and the base layer 12 and the top layer 13 formed on the surface of the coil layer.
c) A guide wire including a linear core wire, a coil layer with a wire spirally wound around at least a part of the outer periphery of the core wire, a coating layer provided on the outer periphery of the coil layer, and the base layer 12 and the top layer 13 formed on the surface of the coating layer.
d) A catheter including a tubular member, and the base layer 12 and the top layer 13 formed on the surface of the tubular member.
e) A catheter including a tubular member, a balloon disposed on one end of the tubular member, and the base layer 12 and the top layer 13 formed on the surface of the balloon.

In an implementation, the elongated medical appliance 1 according to this embodiment may have a configuration different from the above-described configurations (a) to (e), or may be configured as an elongated medical appliance other than guide wires and catheters. The elongated medical appliance 1 may include the base layer 12 and the top layer 13 on at least a part of its surface.

### Method for Producing Elongated Medical Appliance

The method for producing the elongated medical appliance 1 according to this embodiment may include a suitable method that results in the elongated medical appliance 1 having the above-described configuration. In an implementation, from the viewpoint of facilitating formation of the above-described configuration, the elongated medical appliance 1 may be produced according to this embodiment by a production method including a base coat film forming step, a top coat film forming step, and a heating step, described below.

### Base Coat Film Forming Step

First, in the base coat film forming step, a base layer composition containing an isocyanate compound having two or more isocyanate groups is applied onto the substrate 11 provided in the elongated medical appliance 1 to form a base coat film.

As described above, the base layer composition may contain a solvent for facilitating the application. The base layer composition may contain other components such as a resin having at least one of the hydroxy group and the carboxy group.

In the method for applying the base layer composition, e.g., (a coating liquid of) the base layer composition may be applied onto the surface of the substrate 11 using a coater or the like, or using a method in which the substrate 11 is immersed in (a coating liquid of) the base layer composition and then drawn out at a constant speed.

In an implementation, a drying step of volatilizing a solvent or volatile components in the base coat film may be performed after the application of the base layer composition. The drying can be performed e.g. by heating at 50° C. to 150° C. for 60 to 7,200 seconds.

### Top Coat Film Forming Step

Subsequently, in the top coat film forming step, a top layer composition containing a polyamine compound and a copolymer obtained by copolymerizing at least a polymer unit having a hydrophilic structure and a polymer unit having a cyclic carbonate structure is applied onto the surface of the base coat film formed as described above on the side opposite to the substrate 11, to form a top coat film.

As described above, the top layer composition may contain a solvent for facilitating the application. The top layer composition may contain components other than the copolymer and the polyamine compound.

In the method for applying the top layer composition, e.g., (a coating liquid of) the top layer composition may be applied onto the surface of the base coat film using a coater or the like, or using a method in which the substrate 11 coated with the base coat film is immersed in (a coating liquid of) the top layer composition and then drawn out at a constant speed.

In an implementation, a drying step of volatilizing a solvent or volatile components in the top coat film may be performed after the application of the top layer composition. The drying can be performed e.g. by heating at 50° C. to 150° C. for 60 to 7200 seconds.

### Heating Step

Finally, in the heating step, the base coat film and the top coat film formed as described above are heated to form a base layer obtained through curing of the base coat film and a top layer obtained through curing of the top coat film.

On the top coat film, the heating forms a polymer material with copolymers crosslinked with each other in a hydroxyurethane structure (structure including a hydroxy group and a urethane structure), e.g., a polymer material having the structure represented by any of the above-described formulas (1) to (3), by a reaction between the copolymers contained in the top coat film and a polyamine compound, specifically a reaction between the cyclic carbonate group of the copolymer and a polyamine compound. On the interface between the top coat film and the base coat film, at least some of the isocyanate of the isocyanate compound contained in the base coat film react with at least some of the hydroxy groups in the hydroxyurethane structure (e.g., structure represented by the above-described formulas (1) to (3)) in the polymer material contained in the top coat film to covalently bind thereto. This makes it possible to obtain the elongated medical appliance 1 with the substrate 11, the base layer 12, and the top layer 13 sequentially laminated.

When the base layer composition contains a resin having at least one of the hydroxy group and the carboxy group, a reaction between the hydroxy group or carboxy group of the resin and the isocyanate group of the isocyanate compound is also caused by heating in the heating step. Also, when the hydroxy group is present on the surface of the substrate 11, a reaction between the hydroxy group and the isocyanate group of the isocyanate compound is caused by heating in the heating step.

The heating temperature in the heating step may be 50° C. or higher, 60° C. or higher, or 70° C. or higher. This makes it easy for the above-described various reactions to efficiently proceed. The temperature may be 150° C. or lower, 120° C. or lower, or 100° C. or lower. This makes it easy to suppress thermal denaturation of the substrate 11 and the elongated medical appliance 1.

The heating time in the heating step may be 600 seconds or longer, 1,200 seconds or longer, or 1,800 seconds or longer. This makes it easy for the above-described various reactions to efficiently proceed. The time may be 7,200 seconds or shorter, 5,400 seconds or shorter, or 3,600 seconds or shorter. This makes it easy to suppress thermal denaturation of the substrate 11 and the elongated medical appliance 1.

The embodiments explained above are described to facilitate understanding of the disclosed embodiments and not to limit the disclosed embodiments. Thus, the elements disclosed in the above-described embodiments are intended to include all design changes and equivalents belonging to the technical scope of the disclosed embodiments. In some instances, as would be apparent to one of skill in the art as of the filing of the present application, features, characteristics, and/or elements described in connection with a particular embodiment may be used singly or in combination with features, characteristics, and/or elements described in connection with other embodiments unless otherwise indicated.

### EXAMPLES

The disclosed embodiments will be more specifically explained below with reference to Examples and the like, but the scope of the disclosed embodiments is not limited to Examples and the like.

### Example 1

### (1) Preparation of Substrate

A surface of a 0.81 mm diameter SUS304 metal wire was coated with a thermoplastic polyamide elastomer (polyether block amide copolymer, product name “PEBAX 35A NAT-R” manufactured by Arkema S.A.) by a thermal melt extrusion method. This makes it possible to obtain a substrate with the surface of the metal wire coated with a thermoplastic polyamide elastomer having a thickness of about 50 km.

### (2) Formation of Base Layer

An ethoxyethyl acetate solution containing an acrylic resin having a hydroxy group in an amount of 15 mol % based on the total polymer unit was prepared. To the solution, a polyisocyanate (trifunctional HDI nurate) in an amount of 1.25 equivalent per 1 equivalent of the hydroxy group of the acrylic resin was added as an isocyanate compound to obtain a coating liquid of the base layer composition.

The substrate obtained in the step (1) was immersed in the coating liquid and drawn out at a constant speed to form a coat film of the base layer composition on the surface of the substrate. Then, the coat film was dried at 65° C. for 15 minutes using a hot air circulation dryer to form a base layer.

### (3) Formation of Top Layer

To obtain an acrylic copolymer, 40 parts by mass of N-methacryloylaminopropyl-N,N-dimethylammonium-α-N-methylcarboxybetaine (MAMCMB), 50 parts by mass of methoxypolyethylene glycol methacrylate (M90G), and 10 parts by mass of (2-oxo-1,3-dioxolan-4-yl) methylmethacrylate (GCMA) were polymerized by a solution polymerization method. Hereinafter, the polymer is referred to as “Poly (MAMCMB-M90G-GCMA) 40:50:10” in some cases. The copolymer included a polymer unit having a hydrophilic structure derived from MAMCMB and M90G, and a polymer unit having a cyclic carbonate structure derived from GCMA.

The copolymer had a weight-average molecular weight of about 100,000, as measured. Note that the weight-average molecular weight and a weight-average molecular weight of a copolymer described later are standard polystylene-equivalent values measured by a gel permeation chromatography (GPC) method.

The following Formula 11 schematically represents the configuration of the “Poly (MAMCMB-M90G-GCMA) 40:50:10”. Although the “Poly (MAMCMB-M90G-GCMA) 40:50:10” is a random copolymer, Formula 11 presents the above-described three types of structural units arranged in order. In Formula 11, the cyclic carbonate structure is surrounded by a dashed line.

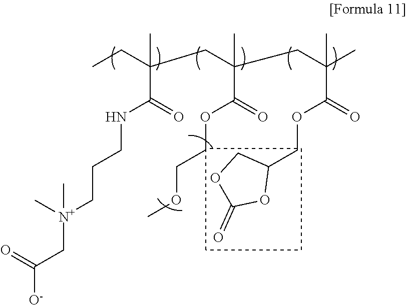

Subsequently, an ethanol solution containing the copolymer produced as described above at a concentration of 20% by mass was prepared. Then, the solution and an ethanol solution containing 5% by mass of hexamethylenediamine (HMDA) as a crosslinking agent were mixed at a weight ratio of 5:3 to obtain a coating liquid of the top layer composition.

The substrate obtained in the step (2) and having the base layer formed on its surface was immersed in the coating liquid of the top layer composition immediately after the mixing and the drawn out at a constant speed to form a coat film of the top layer composition on the surface of the base layer. Then, the coat film was heated at 100° C. for 1 hour using a hot-air circulation dryer. The heating advanced a crosslinking reaction between the copolymer and the crosslinking agent on the coat film of the top layer composition, and at the same time, advanced the reaction between at least some of the hydroxy groups in the crosslinked product formed by the crosslinking reaction and at least some of the isocyanate groups derived from the polyisocyanate in the base layer. As a result, a sample (sample simulating a medical appliance) with a base layer and a top layer sequentially formed on a surface of a substrate was obtained.

### Example 2

To obtain an acrylic copolymer, 90 parts by mass of N,N-dimethylacrylamide (DMAAm) and 10 parts by mass of (2-oxo-1,3-dioxolane-4-yl) methyl methacrylate (GCMA) were polymerized by a solution polymerization method. Hereinafter, the copolymer is referred to as “Poly (DMAAm-GCMA) 90:10” in some cases. The copolymer included a polymer unit having a hydrophilic structure derived from DMAAm, and a polymer unit having a cyclic carbonate structure derived from GCMA. The copolymer had a weight-average molecular weight of about 90,000, as measured.

The following Formula 12 schematically represents the structure of the “Poly (DMAAm-GCMA) 90:10”. Although the “Poly (DMAAm-GCMA) 90:10” is a random copolymer, Formula 12 represents the above-described two types of structural units arranged in order. In Formula 12, the cyclic carbonate structure is surrounded by a dashed line.

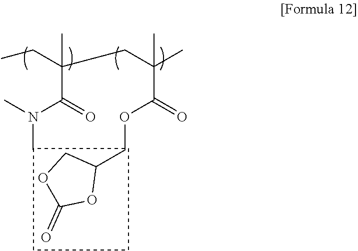

As the copolymer for forming the top layer, a sample with a base layer and a top layer sequentially formed on a surface of a substrate was obtained in the same manner as in Example 1 except that the “Poly (DMAAm-GCMA) 90:10” was used and the solvent for dissolving the copolymer was changed from ethanol to dimethylformamide.

### Example 3

A sample with a base layer and a top layer sequentially formed on a surface of a substrate was obtained in the same manner as in Example 1 except that the thermoplastic polyamide elastomer was replaced by a thermoplastic nylon elastomer (polyamide elastomer, product name “DIAMID L1940”, manufactured by Polyplastics-Evonik Corporation) in preparing the substrate.

### Example 4

A sample with a base layer and a top layer sequentially formed on a surface of a substrate was obtained in the same manner as in Example 2 except that the thermoplastic polyamide elastomer was replaced by a thermoplastic nylon elastomer (polyamide elastomer, product name “DIAMID L1940”, manufactured by Polyplastics-Evonik Corporation) in producing the substrate.

### Comparative Example 1

A sample with a base layer and a top layer sequentially formed on a surface of a substrate was obtained in the same manner as in Example 1 except that the isocyanate compound was not added in preparing the coating liquid of the base layer composition.

### Comparative Example 2

A sample with a base layer and a top layer sequentially formed on a surface of a substrate was obtained in the same manner as in Example 2 except that the isocyanate compound was not added in preparing the coating liquid of the base layer composition.

### Comparative Example 3

A sample with a base layer and a top layer sequentially formed on a surface of a substrate was obtained in the same manner as in Example 3 except that the isocyanate compound was not added in preparing the coating liquid of the base layer composition.

### Comparative Example 4

A sample with a base layer and a top layer sequentially formed on a surface of a substrate was obtained in the same manner as in Example 4 except that the isocyanate compound was not added in preparing the coating liquid of the base layer composition.

### [Test Example 1] (Evaluation of Lubricity)

Each of the samples according to Examples 1 to 4 and Comparative Examples 1 to 4 produced as described above was immersed in physiological saline. Then, each of the samples taken out from physiological saline was subjected to tactile sensation evaluation based on the following criteria, in which each of portions where the top layer and the base layer were provided was pinched with fingertips and rubbed once. Table 1 presents the results as “Initial lubricity” evaluations.
A: Good slippery feeling
B: Insufficient slippery feeling

After the tactile sensation evaluation, the sample was rubbed 10 times in the same manner as described above, and then subjected to the tactile sensation evaluation again. Table 1 presents the results as “Post-rubbing lubricity” evaluations.

Then, for each sample, impressions of the lubricities in a series of evaluations described above are presented in the column “Surface state” in Table 1. Table 1 also presents coating materials for the substrate of each sample, the presence or absence of the isocyanate compound in the base layer, and the type of the copolymer in the top layer.

TABLE 1

| | | Isocyanate | | | Lubricity | |
|---|---|---|---|---|---|---|
| | Coating material | compound in | Copolymer in | Initial | after | |
| | of substrate | base layer | top layer | lubricity | rubbing | Surface state |
| Example 1 | Thermoplastic | Yes | Poly(MAMCMB- | A | A | Lubricity was |
| | polyamide | | M90G-GCMA) | | | maintained at all |
| | elastomer | | 40:50:10 | | | times |
| Example 2 | Thermoplastic | Yes | Poly(DMAAm- | A | A | Lubricity was |
| | polyamide | | GCMA) 90:10 | | | maintained at all |
| | elastomer | | | | | times |
| Example 3 | Thermoplastic | Yes | Poly(MAMCMB- | A | A | Lubricity was |
| | nylon elastomer | | M90G-GCMA) | | | maintained at all |
| | | | 40:50:10 | | | times |
| Example 4 | Thermoplastic | Yes | Poly(DMAAm- | A | A | Lubricity was |
| | nylon elastomer | | GCMA) 90:10 | | | maintained at all |
| | | | | | | times |
| Comparative | Thermoplastic | None | Poly(MAMCMB- | A | B | Lubricity gradually |
| Example 1 | polyamide | | M90G-GCMA) | | | disappeared |
| | elastomer | | 40:50:10 | | | |
| Comparative | Thermoplastic | None | Poly(DMAAm- | A | B | Lubricity gradually |
| Example 2 | polyamide | | GCMA) 90:10 | | | disappeared |
| | elastomer | | | | | |
| Comparative | Thermoplastic | None | Poly(MAMCMB- | A | B | Lubricity gradually |
| Example 3 | nylon elastomer | | M90G-GCMA) | | | disappeared |
| | | | 40:50:10 | | | |
| Comparative | Thermoplastic | None | Poly(DMAAm- | A | B | Lubricity gradually |
| Example 4 | nylon elastomer | | GCMA) 90:10 | | | disappeared |

As is clear from Table 1, the samples according to Examples exhibited good lubricity. The samples according to Examples exhibited good lubricity even after rubbing 10 times. From this, it is presumed that the adhesiveness between the base layer and the top layer is improved by the isocyanate compound contained in the base layer, and as a result, the lubricity can be desirably maintained.

### Example 5

A sample with a base layer and a top layer sequentially formed on a surface of a substrate was obtained in the same manner as in Example 1 except that an SUS304 plate was used as a substrate and the base layer and top layer were sequentially formed on one side of the plate using a bar coater.

### Comparative Example 5

A sample with a base layer and a top layer sequentially formed on a surface of a substrate was obtained in the same manner as in Example 5 except that the isocyanate compound was not added in preparing the coating liquid of the base layer composition.

### [Test Example 2] (Evaluation of Adhesiveness)

In the samples according to Example 5 and Comparative Example 5, the surface on the side with the base layer and the top layer was subjected to a cross-cut test (Japanese Industrial Standards (JIS) K5600-5-6: in 1999) to evaluate the adhesiveness of the base layer and top layer to the substrate.

Specifically, the base layer and the top layer were slit in a lattice shape at an interval of 1 mm using a cutter, a transparent adhesive tape was stuck to the layers and then peeled off, and the state of the lattice was observed to confirm the peeled state of the coating film. The details of the test conditions were in accordance with JIS K5600-5-6: in 1999.

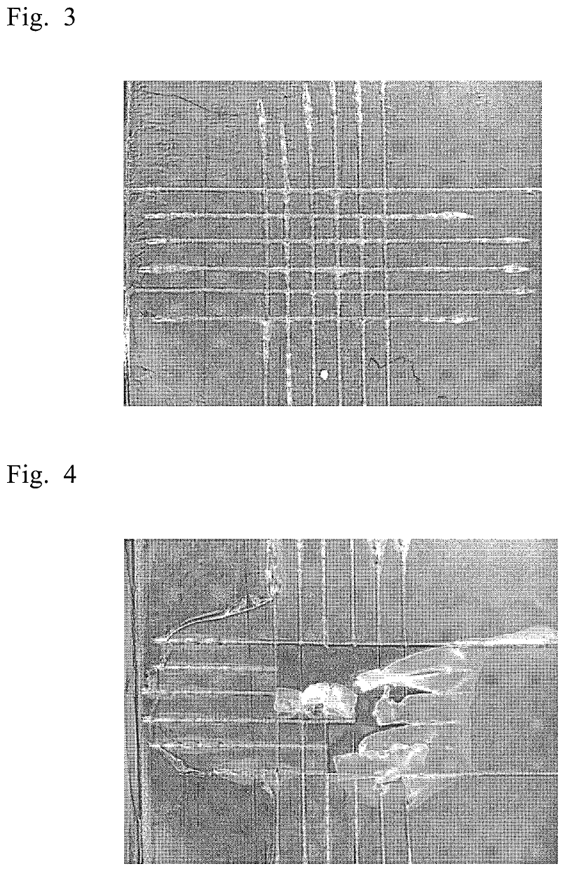

The test results are presented in FIG. 3 and FIG. 4. FIG. 3 is an image obtained by photographing a slit portion in the sample according to Example 5. FIG. 4 is an image obtained by photographing a slit portion in the sample according to Comparative Example 5.

As is clear from FIG. 3, in the sample according to Example 5 (sample for which the isocyanate compound was used in forming the base layer), the base layer and the top layer were not peeled off in all of the lattices (the evaluation result defined in JIS: Category 0).

On the other hand, as is clear from FIG. 4, in the sample according to Comparative Example 5 (sample for which the isocyanate compound was not used in forming the base layer), the base layer and the top layer were peeled off in many test areas (the evaluation result defined in JIS: Category 4). Particularly, the sample was also confirmed to have peeling on the interface between the substrate and the base layer and furthermore peeling on the interface between the base layer and the top layer.

From the above results, it was found that the use of the isocyanate compound in forming the base layer improved not only the adhesiveness on the interface between the substrate and the base layer but also the adhesiveness on the interface between the base layer and the top layer. Such an improvement in adhesiveness is presumed to result from the reaction between the isocyanate group of the isocyanate compound in the base layer and the hydroxy group of the polymer material in the top layer, and the covalent bond between these groups.

### Technical Problems

When a lubricating coating film is provided on the substrate as in the medical appliance disclosed in Patent Literature 1, the adhesiveness between the substrate and the coating film is insufficient, and thus there has been a problem of the coating film prone to breakage, exfoliation, depletion, or the like.

The disclosed embodiments have been made in view of such circumstances, and an object of the disclosed embodiments is to provide an elongated medical appliance excellent in lubricity and adhesiveness between a substrate and a coating film, and a method for producing the elongated medical appliance.

### INDUSTRIAL APPLICABILITY

The elongated medical appliance according to the disclosed embodiments is suitable e.g. as a guide wire or a catheter.

### DESCRIPTION OF REFERENCE NUMERALS

1. Elongated medical appliance
11. Substrate
12. Base layer
13. Top layer

1. An elongated medical appliance, comprising:
a substrate,
a base layer on the substrate, and
a top layer on the base layer and on a side opposite to the substrate,
wherein the top layer includes a polymer material with a copolymer comprising a polymer unit having a hydrophilic structure and being crosslinked by a structure represented by one of following formulas (1) to (3):
*—CH(OH)—CH(R¹)—O—C(═O)—NH—R²—NH—C(═O)—OCH(R¹)—CH(OH)—* (1)
*—CH(OH)—CH(R¹)—O—C(═O)—NH—R²—NH—C(═O)—OCH(CH(R¹)—OH)—* (2)
*—CH(CH(R¹)—OH)—O—C(═O)—NH—R²—NH—C(═O)—OCH(CH(R¹)—OH)—* (3)
wherein, * is a linking point to an adjacent atom, each R¹ is the same as or different from each other, and represents a hydrogen atom, a linear alkyl group having 1 or more carbon atoms, or a branched alkyl group having 3 or more carbon atoms, each R² is the same as or different from each other, and represents an alkylene group having 1 or more carbon atoms, a divalent cycloaliphatic hydrocarbon group having 3 or more carbon atoms and comprising a cycloaliphatic structure, or a divalent aromatic group having 6 or more carbon atoms and comprising an aromatic ring structure, in which the alkylene group, the cycloaliphatic hydrocarbon group, or the aromatic group optionally includes a divalent group represented by —NR³— between carbon atoms, in which R³ is hydrogen atom or an alkyl group having 1 to 8 carbon atoms,
wherein the base layer includes an isocyanate compound having two or more isocyanate groups, and
wherein at least some isocyanate groups of the isocyanate compound react with at least some hydroxy groups in the structures represented by the above formulas (1) to (3) in the polymer material and are covalently bonded thereto.

2. The elongated medical appliance according to claim 1, wherein the base layer is prepared from a base layer composition containing the isocyanate compound and a resin having a hydroxy group or a carboxy group.

3. The elongated medical appliance according to claim 1, wherein the copolymer includes a polymer unit having a hydrophilic structure and a polymer unit having a cyclic carbonate structure.

4. The elongated medical appliance according to claim 1, wherein the hydrophilic structure comprises a betaine structure, an amide structure, an alkylene oxide structure, or a lactam structure.

5. A method for producing an elongated medical appliance, the method comprising:
a base coat film forming step of applying a base layer composition containing an isocyanate compound having two or more isocyanate groups onto a substrate for the elongated medical appliance to form a base coat film;
a top coat film forming step of applying a top layer composition containing a polyamine compound and a copolymer including a polymer unit having a hydrophilic structure and a polymer unit having a cyclic carbonate structure onto a surface of the base coat film on a side opposite to the substrate to form a top coat film; and
a heating step of heating and curing the base coat film and the top coat film to form a base layer of the cured base coat film and a top layer of the cured top coat film.
